(19) United States
(12) Patent Application Publication
(10) Pub. No.: US 2024/0374205 A1
(43) Pub. Date: Nov. 14, 2024

(54) SYSTEM AND METHOD FOR ANALYZING USER PHYSICAL CHARACTERISTICS AND PRESCRIBING TREATMENT PLANS TO THE USER

(71) Applicant: METADOC.AI LLC, Las Vegas, NV (US)

(72) Inventors: Armia Abdo, Mission Viejo, CA (US); Bruce Hoffman, Mammoth Lakes, CA (US); Nathaniel Joseph Gonzales, Henderson, NV (US)

(73) Assignee: METADOC.AI LLC, Las Vegas, NV (US)

(21) Appl. No.: 18/782,618

(22) Filed: Jul. 24, 2024

Related U.S. Application Data
(63) Continuation of application No. 17/725,925, filed on Apr. 21, 2022.
(60) Provisional application No. 63/177,769, filed on Apr. 21, 2021.

(51) Int. Cl. A61B 5/00; A63B 24/00; G16H 20/30; G16H 50/30

(52) U.S. Cl. CPC A61B 5/45; A63B 24/0062; A63B 24/0075; G16H 20/30; G16H 50/30; A63B 2024/0065

(57) ABSTRACT

Technologies for evaluating a user's musculoskeletal health and prescribing treatment plans for the user based on the musculoskeletal health is provided. The disclosed techniques include a computing system receiving input data from a client device. The system formatting the input data to generate one or more sets of user characteristic data values and determining initial levels of user musculoskeletal health for the user based upon the sets of user characteristic data values. The system selects a set of treatment plans for the user based on the initial levels of user musculoskeletal health. The system presents the set of treatment plans to the user using media that allows the user to interactively perform exercises prescribed in the treatment plan.

### CROSS REFERENCE TO RELATED APPLICATIONS; PRIORITY CLAIM

The application is a continuation of nonprovisional application Ser. No. 17/725,925, filed on Apr. 21, 2022, which claims benefit to Provisional Application 63/177,769, filed on Apr. 21, 2021. The entire contents of applications Ser. No. 17/725,925 and 63/177,769 are hereby incorporated by reference as if fully set forth herein, under 35 U.S.C. § 119(e).

### TECHNICAL FIELD

The present disclosure generally relates to analyzing user input data and determining treatment for the user based on user interactions with a computing device.

### BACKGROUND

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

Conventional healthcare plans treat injuries to the muscular system by prescribing pain medications or by physical therapy. Pain medications are generally directed towards pain management and do not treat the underlying cause of the pain. For example, if a patient has back pain caused by a slipped disc, prescription of pain medication will treat the back pain by blocking the pain receptors in the back but do not address the cause of the back pain, the slipped disk and the mechanical stressors that influence it.

Physical therapy is another approach to treat injuries that limit a patient's ability to move and perform day-to-day physical functions. Conventional physical therapy involves a physical therapist physically examining the patient to identify deficiencies in a patient's movement in order to identify one or more physical ailments that may need treatment. The treatment prescribed may involve a series of exercises and stretches designed to treat problem areas in the body of the patient. For example, a physical therapist may prescribe a series of back stretches for lower back pain caused by a slipped disc. The treatment plan may include having the patient perform the prescribed exercises in front of the physical therapist while the patient is at the physical therapist's office. Additionally, the prescription may require the patient to perform the prescribed exercises at home, unsupervised, until the next in-person therapy session. However, exercises performed by the patient, without supervision, may cause the patient to reinjure themselves if they are performing the exercise incorrectly. Additionally, if the patient suffers a different physical impairment as a result of in-home exercises, then performing the originally prescribed exercises, unsupervised, may cause additional pain or discomfort to existing physical injuries or may cause new injuries.

One possible solution would be to only perform prescribed physical exercise while supervised by the physical therapist. However, in-person supervision may not be possible due to the lack of availability of physical therapists, the required frequency for performing such exercises, and/or the cost of having a physical therapist supervise for each session.

### BRIEF DESCRIPTION OF THE DRAWINGS

In the drawings:

FIG. 1 illustrates an example system for analyzing user input data, determining a set of treatment plans, and presenting the treatment plans to the user, in accordance with an embodiment.

FIG. 2 depicts an example flowchart for analyzing user input data, determining levels of musculoskeletal health for the user and risk of injury, and selecting a set of treatment plans based on the user's musculoskeletal health and risk of injury, in accordance with an embodiment.

FIG. 3 depicts an example flowchart for generating and displaying an interactive video that contains a digital representation of the user performing the exercises, in accordance with an embodiment.

FIG. 4 is a block diagram that illustrates a computer system upon which an embodiment of the invention may be implemented.

FIG. 5 illustrates an example system for analyzing user input data, determining a set of treatment plans, and presenting the treatment plans to the user, in accordance with an embodiment.

FIG. 6 depicts an example flowchart for calculating postural health values for anatomical landmarks identified for a user, in accordance with an embodiment.

FIG. 7 depicts an example image of a user's body and example anatomical landmarks used to determine postural health values for the user, according to an embodiment.

### DETAILED DESCRIPTION

In the following description, for the purposes of explanation, numerous specific details are set forth in order to provide a thorough understanding of the present invention. It will be apparent, however, that the present invention may be practiced without these specific details. In other instances, well-known structures and devices are shown in block diagram form in order to avoid unnecessarily obscuring the present invention.

### General Overview

The appended claims may serve as a summary of the invention.

In an embodiment, systems and methods determine a set of treatment plans for a user based upon a determined level of musculoskeletal health and risk of injury to the user. For example, the system may use one or more client computing devices to determine the musculoskeletal health and risk of injury to the user. Based upon the user's level of musculoskeletal health and risk of injury, the system may select one or more treatment plans for the user. A treatment plan may include, but is not limited to, physical exercise instructions, instructions on how to perform physical treatment on one or more parts of the user's body, prescribed medications, education on physical exercises and movements, user diagnosis reports, as well as physical fitness goals and methods to track a user's treatment. For example, the system may determine that the user should perform a series of stretching exercises to treat the user's injured back. Upon determining the one or more treatment plans, the system may present the one or more treatment plans to the user. For instance, the system may present stretching exercises in the form of an instructional video. In another example, the system may present the stretching exercises using an interactive video that records and displays, in real-time, the user's movement as the user performs the stretching exercise.

Musculoskeletal health may describe the overall health of a region of a user's body, such as excellent musculoskeletal health, above average musculoskeletal health, average musculoskeletal health, and poor musculoskeletal health. Postural health may describe the overall health of a region of the body and/or may represent a numerical value that represents the musculoskeletal health. For example, a postural health value of 4 may represent excellent musculoskeletal health. For the purposes of this disclosure, the terms musculoskeletal health and postural health may be used interchangeably.

### System Overview

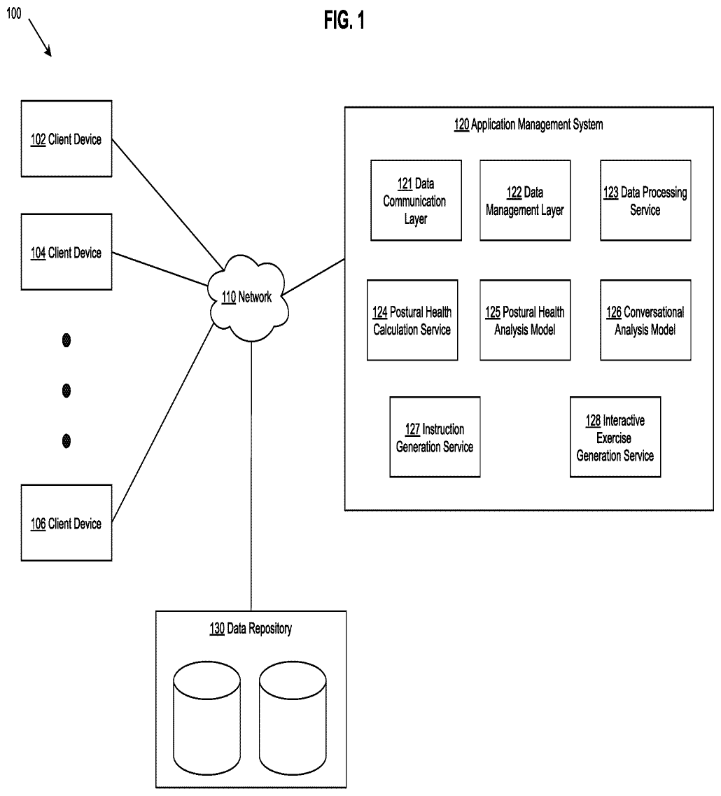

FIG. 1 illustrates an example system for analyzing user input data, determining a set of treatment plans, and presenting the treatment plans to the user, in accordance with an embodiment. System 100 generally determines one or more treatment plans for a user and presents the treatment plans to the user in a manner that allows the user to track their progress during physical rehabilitation. The example system 100 is conceptually described herein as comprising client devices 102-106, an application management system 120, and a data repository 130. System 100 may represent one example arrangement and other system arrangements may include more or less components than what is depicted in system 100.

A user may represent a patient seeking rehabilitation treatment from a physical therapist or other medical professional. Users may interact with system 100 using client devices 102-106. In an embodiment, client devices 102-106 may represent computing devices including, but not limited to, desktop computers, laptop computers, tablet computers, wearable devices, webcam enabled computers, video game consoles, smartphones, and any other computing device that is enabled with input/output support. In another embodiment, client devices 102-106 may represent peripheral computing devices configured to receive input from a user and send the input to another computing device and/or the application management system 120 via network 110. For example, a user may interact with system 100 using client device 102, which represents a laptop with a webcam. Client device 102 may be implemented to capture video of the user as the user stands, stretches, or demonstrates their physical limitations due to an injury. For instance, the client device 102 may capture video of the user standing naturally. Client device 102 may then send the captured video images, via network 110, to the application management system 120 for processing. Further details on client devices 102-106 are described in the CLIENT DEVICES section herein.

### Application Management System

In an embodiment, the application management system 120 may be implemented to receive user input from client devices 102-106, analyze the user input, and determine a set of treatment plans for the user based on the received user input. The application management system 120 may include one or more computing devices (such as a rackmount server, a router computer, a server computer, a personal computer, a mainframe computer, a laptop computer, a tablet computer, a desktop computer, etc.), data stores (e.g., hard disks, memories, databases), networks, software components, and/or hardware components that may be used implement application services for receiving and processing user input.

In an embodiment, the application management system 120 may be communicatively coupled to client devices 102-106 and data repository 130 via network 110. Network 110 may broadly represent one or more local area networks, wide area networks, global interconnected internetworks such as the public internet, or a combination thereof. The application management system 120 includes a data communication layer 121, a data management layer 122, a data processing service 123, a postural health calculation service 124, a postural health analysis model 125, a conversational analysis model 126, an instruction generation service 127, and an interactive exercise generation service 128. Other embodiments of the application management system 120 may include more or less components than depicted in FIG. 1.

In an embodiment, the data communication layer 121 may be implemented to receive data values from client devices 102-106 and the data repository 130 and send data values to client devices 102-106 and the data repository 130. “Layer,” in this context, refers to any combination of electronic digital interface circuits, microcontrollers, firmware such as drivers, and/or computer programs or other software elements. The data communication layer 121 may be programmed or configured to perform input/output interfacing functions including, but not limited to, receiving data values from client devices 102-106, sending data values to the data management layer 122 and the data processing service 123. The data communication layer 121 may be programmed or configured to send and receive data requests to the data repository 130.

In an embodiment, the data management layer 122 may be programmed or configured to manage read operations and write operations involving the data repository 130 and other functional elements of system 100, including queries and result sets communicated between the functional elements of the application management system 120 and the data repository 130. Examples of data management layer 122 include JDBC, SQL server interface code, and/or HADOOP interface code, among others. The data repository 130 may comprise a database. As used herein, the term “database” may refer to either a body of data, a relational database management system (RDBMS), or to both. As used herein, a database may comprise any collection of data including hierarchical databases, relational databases, flat file databases, object-relational databases, object-oriented databases, distributed databases, and any other structured collection of records or data that is stored in a computer system. Examples of RDBMS's include, but are not limited to including, ORACLE®, MYSQL, IBM® DB2, MICROSOFT® SQL SERVER, SYBASE®, and POSTGRESQL databases. However, any database may be used that enables the systems and methods described herein.

In an embodiment, the data management layer 122 may implement data privacy standards according to the Health Insurance Portability and Accountability Act (HIPAA) to store data values within the data repository 130. The data values stored within the data repository may include any user input such as video frames, audio recordings, written input into a chat box or a user interface form, or any other method to collect user input.

In an embodiment, the data processing service 123 may be implemented to generate one or more sets of user characteristic data values for determining musculoskeletal health of the user. Specifically, the data processing service 123 may analyze received user input data values to identify anatomical landmarks of the user. For example, if the user input data values represent video frames depicting the user standing upright and facing client device 102, then the data processing service 123 may analyze the received video frames to identify anatomical landmarks of the user, such as the user's temples, ears, jawline, shoulders, collar bones, elbows, hips, and so on. Each of these anatomical landmarks are then assigned location coordinates that may be used to determine whether the user has healthy posture in various positions. For instance, if the distance between the user's right temple and the user's right shoulder is much less than the distance between the user's left temple and the user's left shoulder, then the application management system 120 may determine that the user's right shoulder muscles are strained and may require physical treatment. Location coordinates for each of the anatomical landmarks of the user may be stored in the data repository 130.

In an embodiment, the data processing service 123 may be implemented to format any type of user input data and convert it into data suitable for analysis by the postural health calculation service 124, the conversational analysis model 126, and any other internal or external data analysis service. For example, the data processing service 123 may process user input data including, but not limited to, digital images, one or more video frames, audio recordings, written input into a chat box or a user interface form, imported medical records, or any other user data input. The data processing service 123 may format the user input data by encapsulating relevant data values within data objects such as XML. Formatted data values within an XML object may be analyzed by different services in order to select a set of treatment plans customized to the user. Formatting data values is not limited to using XML objects, the data processing service 123 may be programmed or configured to implement any data object format for storing data values.

### Postural Health Calculation and Analysis

In an embodiment, the postural health calculation service 124 may be implemented to calculate postural health values for one or more areas on the user's body based upon the location coordinates of the anatomical landmarks of the user. Postural health values may represent a set of metrics used to determine whether treatment should be prescribed to the user and which types of treatment should be prescribed to the user. In an embodiment, postural health values may be used to categorize the musculoskeletal health of a portion of a user's body. For example, postural health values for different portions of the body may be categorized as:

| Categories of | |
|---|---|
| Musculoskeletal Health | Interpretation |
| Excellent Musculo- | Injury and symptoms are unlikely |
| skeletal Health | |
| Good Musculoskeletal | Symptoms are minimal and intermittent. |
| Health | Chance of injury is minimal. |
| Fair Musculoskeletal | Symptoms are moderate and likely chronic. |
| Health | Chance of injury is moderate. |
| Poor Musculoskeletal | Symptoms are significant and chronic. |
| Health | Symptoms may involve other systems of the |
| | body. Chance of injury is high. |

In an embodiment, the postural health values may be converted to a 0-100% scale, where postural health percentage values may be categorized based on categorization thresholds. For instance, “Excellent Musculoskeletal Health” may include postural health percentage values between 80-100%, “Good Musculoskeletal Health” may include postural health percentage values between 70-79%, “Fair Musculoskeletal Health” may include postural health percentage values between 60-69%, and “Poor Musculoskeletal Health” may include postural health percentage values between <60%. In other embodiments, postural health percentage values may be categorized differently for different portions of the body. For example, postural health values may be categorized into a series of buckets, such as buckets 1-4 where bucket 1 may represent “Excellent Musculoskeletal Health” and bucket 4 may represent “Poor Musculoskeletal Health”.

In an embodiment, the postural health calculation service 124 may calculate postural health values and risk of injury by determining position and movement of the anatomical landmarks and comparing the positions and movements to other anatomical landmarks of the user and other users. Each anatomical landmark may be labeled using the following format, (anatomical landmark ID, side of body, 3d resting position), where (1,1,1) represents the left temple. The anatomical landmark for the temple may be represented by ID=1, the left side of the body equals 1, and the right side of the body equals 2, and a first 3d resting position equals 1 and a second 3d resting position equals 2. Each anatomical landmark label may be assigned (x, y, z) coordinate values that represent degrees in a rotational matrix away from the center. Coordinate values for anatomical landmarks may then be compared to each other. For instance, when evaluating body symmetry, the coordinate values of a left body portion may be compared to the coordinate values of a right body portion. If the left body portion and the right body portion are symmetrical, then the difference between the values would equal zero. If, however, the left body portion and the right body portion are not symmetrical then the difference between the values would equal a non-zero value. Treatment options may be selected depending on how asymmetrical a user's left body portion is when compared to the user's right body portion.

In other embodiments, the postural health calculation service 124 may calculate postural health values using other types of formatted data. For example, the data processing service 123 may analyze medical records for a user to generate data values representing physical characteristics of the user, such as their height, weight, and measurements of specific portions of the body. The postural health calculation service 124 may be configured to compare locations of anatomical landmarks based on the data values generated from the medical records.

In an embodiment, the postural health analysis model 125 may be implemented to select one or more treatment plans for the user based upon the postural health values calculated by the postural health calculation service 124. The postural health analysis model 125 may be programmed or configured to implement a machine-learned model for selecting one or more treatment plans based on the calculated postural health values. The postural health analysis model 125 may generate and train the machine-learned model to select the one or more treatment plans for a user based upon comparing the user's postural health values to the user's previously measured postural health values as well as historical postural health values of other users that have the same or similar physical features to the user in question. For example, if the user is a 45-year-old male who weighs 200 lbs. and is 6 feet tall, then the postural health analysis model 125 may compare historical postural health values from other users that are in the same age, weight, and height range as the user in question. The postural health analysis model 125 may also consider other user characteristics such as the user's level of activity, preferred physical activities, known health issues, and any other user characteristic that may be helpful in determining treatment plans for the user.

In an embodiment, the machine-learned model may be a regression model, such as a linear regression model or a logistic regression model, where the input into the model is a set of data values including calculated postural health values for the user as well as a set of user characteristics that describe the user's physical characteristics, pain levels, pain frequency, health, and injury history. The output from the model may be one or more selected treatment plans, where each treatment plan may include specific instructions on how to perform the prescribed exercises and their frequency. For example, if the user is a 45-year-old male that plays basketball and suffers from chronic low-back pain, then input into the model may include a set of data values comprising the user's physical characteristics, health history, physical activities (e.g., playing basketball), and the user's calculated postural health values. The postural health analysis model 125 may utilize historical postural health values from other users that are similar to the current user to select one or more treatment plans. In an embodiment, the postural health analysis model 125 may also consider a user's geo-location when selecting historical postural health data and when selecting treatment plans based on the historical postural health data. For example, if a user is suffering from knee pain and lives in a cold environment, such as Minnesota, then the postural health analysis model 125 may evaluate the user against historical postural health data from other users in similar climates.

In an embodiment, the postural health analysis model 125 may receive additional user data corresponding to a user's behavioral tendencies, notification preferences, and motivation preferences in order to customize the treatment plans. For example, the application management system 120 may present the user with a series of questions related to their notification preferences and motivation preferences. The postural health analysis model 125 may receive the notification preferences and motivation preferences and use the preferences to customize how the application management system 120 schedules exercises for the user, reminds the user of their exercise schedule, and motivates the user to participate in the prescribed exercises. In an embodiment, the postural health analysis model 125 may generate notifications for the user in response to detecting certain user activities. For example, if the user's smart device, such as a smart watch, detects that the user is performing walking exercises or activities, the postural health analysis model 125 may generate and send a notification that includes walking or other exercise tips to improve and/or maintain a user's good posture and/or exercise habits.

In an embodiment, the postural health analysis model 125 may implement any machine learning technique to generate the machine-learned model. Examples of machine learning algorithms implemented by postural health analysis model 125 may include, but are not limited to, logistic regression, linear regression, random forest, decision tree learning, association rule learning, artificial neural network, support vector machines, and/or Bayesian networks. Embodiments are not limited to any particular type of machine learning technique or algorithm.

The postural health calculation service 124 and the postural health analysis model 125 are illustrated as part of one embodiment of the application management system 120. In other embodiments of the application management system 120, the application management system 120 may include one or more health calculation services as well as one or more health analysis models. The one or more health calculation services may be configured to calculate data health values directed to evaluating ailments related to vision, the integumentary system, the nervous system, the endocrine system, the lymphatic and immune system, the respiratory system, the digestive system, the urinary system, and the reproductive system. The one or more health analysis models may be implemented to analyze the data health values and determine whether the user may need further treatment or additional consultations from various health care professionals or specialists related to vision, the integumentary system, the nervous system, the endocrine system, the lymphatic and immune system, the respiratory system, the digestive system, the urinary system, the reproductive system, mental health, and any other system or body function. Additional embodiments of the application management system 120 are described in the HEALTH ANALYSIS MODELS section herein.

### Speech Analysis

The data communication layer 121 may receive different types of user input data, such as inputted text from a chat box or an online form. The data processing service 123 may format inputted text based on the type of user responses and/or the type of online form presented. For example, if the chat box prompted the user for their injury history, then the received text from the user may be formatted into an XML data object, or any other format, which labels the responses as injury history. In an embodiment, the conversational analysis model 126 may be implemented to analyze inputted user responses and identify relevant keywords and phrases. For example, if the chat box asks the user a question about their level of pain experienced, the conversational analysis model 126 may parse and identify relevant user responses to determine the user's level of pain experienced. User responses may be subjective such as “it hurts a lot” or “it's painful but manageable”. The conversational analysis model 126 may determine the user's level of pain by evaluating the user's subjective response based on how the user uses descriptive words in conversation. In an embodiment, the conversational analysis model 126 may be programmed or configured to implement a machine-learned model for analyzing and interpreting user text responses to prompted questions. The conversational analysis model 126 may generate and train the machine-learned model to identify keywords and phrases based upon conversational tendencies of the user. For example, the machine-learned model may analyze user demographics, such as age, gender, race, marital status, number of children, occupation, annual income, education level, living status, geographical location, and any other user characteristics that may be used to identify and interpret the user's phrases, slang, and overall emotion in their responses. Additionally, historical responses from the user and other responses from similar users may be used to aid in interpreting the user's response. In an embodiment, the output of the machine-learned model may be formatted data values that reflect an interpretation of the user's input. For example, if the user responded to a question about knee pain with “it's painful but manageable”, then the conversational analysis model 126 may use user demographics and historical conversation data to interpret “it's painful but manageable” from the user to mean that the knee pain is a 6 out of 10. Other users who may respond similarly to the same question, may be interpreted to have a different level of pain based upon their user demographics and historical conversation data. The output from the conversational analysis model 126 may be used as additional input for the postural health analysis model 125 to select the one or more treatment plans for the user.

### Presenting Treatment Plans

Upon determining one or more treatment plans for the user, the postural health analysis model 125 may send the one or more treatment plans to the instruction generation service 127 for presenting to the user. In an embodiment, the instruction generation service 127 may be programmed or configured to present the one or more treatment plans to the user in various presentation formats including, but not limited to, video instruction, interactive video instruction, written instructions, audio instructions, augmented reality (AR) instructions, virtual reality (VR) instructions, and any other media format. For example, the instruction generation service 127 may present the one or more treatment plans to the user as a series of videos that may be presented to the user using client devices 102-106. The series of videos may be displayed within a customized user interface with an embedded video player. In another example, if the user is interacting with the application management system 120 via an audio connection, then the instruction generation service 127 may format and present the one or more treatment plans as audio instruction, where the user listens to the audio instruction and performs the prescribed exercises based on the audio instruction. If the user requests the treatment plans in the form of text and pictures, then the instruction generation service 127 may format and present the one or more treatment plans as a list of instructions that the user may print or view using a word processing program.

In an embodiment, the interactive exercise generation service 128 may be programmed or configured to implement one or more interactive videos for the user based on the one or more treatment plans selected. Interactive videos may represent a video session where the user is displayed in real-time or near real-time within the interactive video. For example, the interactive video may be displayed by client device 102, which is a computer enabled with a webcam. Client device 102 may display the video on the display screen, simultaneously capture video of the user, and superimpose a representation of the user onto the video displayed on the display screen. The interactive video may display alerts when the user is either performing the displayed exercise correctly or incorrectly. For example, if the user is instructed to perform a forward lunge, then the interactive video may display a green check mark when the user's anatomical landmarks are positioned correctly when performing the lunge. If, however, the user does not bend his knees far enough, then the interactive video may display a message encouraging the user to bend his knees further. Interactive messages and icons may be text based, symbol based, or audio based. An example of an audio message may be an audible ding, cheer, or speech such as “good job”, “a little further”, or “almost, try again.”

In an embodiment, detected user movements during performance of an exercise may be received by the data communication layer 121. The data processing service 123 may format the received data and the data management layer 122 may store the formatted data in the data repository 130. In an embodiment, the postural health analysis model 125 may periodically review stored data of the user performing the prescribed exercises to determine whether the user is progressing as expected or if there is a deviation in the user's rehabilitation. If there is a deviation in the rehabilitation, such as the user is progressing slowly, is suffering a setback, or the user is improving at a faster than expected pace, then the postural health analysis model 125 may adjust the treatment plans accordingly. For example, if the user repeatedly fails to complete the prescribed exercises, then the postural health analysis model 125 may modify the number of repetitions or the degree of movement for the user in order to help the user improve. Alternatively, if the user is improving at a faster than expected pace, then the postural health analysis model 125 may modify and add additional exercises or repetitions to help the user improve more quickly.

### Client Devices

Referring back to FIG. 1, client devices 102-106 may represent computing devices including, but not limited to, desktop computers, laptop computers, tablet computers, wearable devices, webcam enabled computers, video game consoles, smartphones, and any other computing device that is enabled with input/output support. In an example, client devices 102-106 may represent computing devices enabled with a digital camera. The digital camera may be programmed or configured to collect visual data, in the form of still images or video frames, for the purposes of identifying shapes, movements, and anatomical landmarks of the user. An example of identifying relevant shapes may include identifying specific facial expressions that may indicate whether the user is experiencing pain and to what degree.

In an example, client devices 102-106 may represent computing devices enabled with audio capturing equipment such as one or more microphones. The one or more microphones may be used to detect speech from the user. Detected speech may be analyzed by the application management system 120 to identify any audio cues indicating a state of the user. For example, a microphone on client device 102 may detect an audible grunt when the user is stretching his hamstrings. The application management system 120 may receive the audio cue from client device 102 and may determine that the user is experiencing more pain than usual when performing a prescribed exercise.

In an example, client devices 102-106 may represent computing devices enabled with a display screen for displaying an interactive graphical user interface. The interactive graphical user interface may be used to prompt the user with a form or chat box. The user may provide user input to the graphical user interface. The user input may be sent to the application management system 120 for processing. For example, user input inputted into a chat box may be sent to the application management system 120. The conversational analysis model 126 may receive the user input and may identify and interpret various responses from the user.

In an example, client devices 102-106 may represent computing devices enabled with touch feedback, such as wearable devices. Wearable devices may be used to gather information from contact with the user's body. Examples of information gathered from a wearable include, but are not limited to, temperature, electrical currents, body tension, heart rate, blood pressure, and any other user vital signs. Wearable devices may be used during interactive exercises to alert the user when they are performing specific exercises correctly. For example, if the user is asked to perform 10 toe touches, then the gyroscope enabled in the wearable device may be used to determine whether the user is fully bending over to touch their toes. Alerts may include a single feedback buzz when the user successfully performs a toe touch and a double feedback buzz when the user unsuccessfully performs the toe touch.

In an example, client devices 102-106 may represent computing devices enabled with olfactory sensors. The olfactory sensors may be used to detect specific odors from the user that may indicate a presence of a potential pathogen. The presence of potential pathogens may cause the application management system 120 to modify the prescribed treatment plan to account for any potential illnesses or infections that the user may be experiencing.

In other examples, any computing device programmed or configured to detect user behavior may be used to alert the application management system 120 of the user's physical tendencies, which may be clues to different physical ailments the user may be experiencing. For example, client devices 102-106 may be used to detect how a user inputs keystrokes, writes by hand, walks normally, the way a user uses the mouse, and any other repetitive movements that may be a clue about the user's condition.

Use cases for client devices 102-106 are further illustrated in examples listed in Appendix A. The use cases described in Appendix A represent only a sample of the potential ways client devices 102-106 may be used with the application management system 120.

### Health Analysis Models

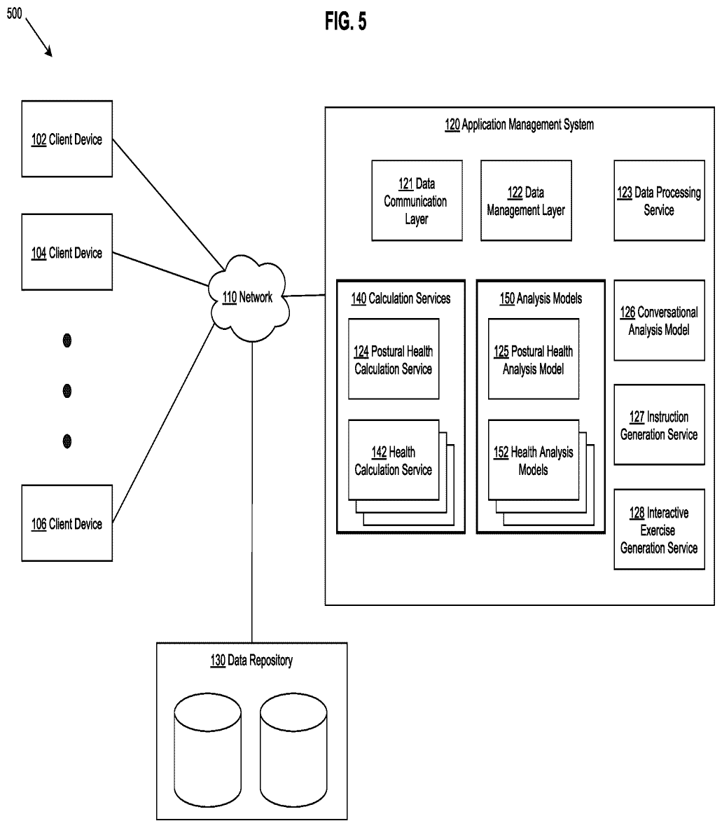

FIG. 5 illustrates another example system for analyzing user input data, determining a set of treatment plans, and presenting the treatment plans to the user, in accordance with an embodiment. System 500 generally determines one or more treatment plans for a user and presents the treatment plans to the user in a manner that allows the user to track their progress during the user's recovery. The example system 500 is conceptually described herein as comprising client devices 102-106, the application management system 120, and the data repository 130. System 500 may represent one example arrangement and other system arrangements may include more or less components than what is depicted in system 500.

In an embodiment, the application management system 120 may be implemented to receive user input from client devices 102-106, analyze the user input, and determine a set of treatment plans for the user based on the received user input. The application management system 120 may include one or more computing devices (such as a rackmount server, a router computer, a server computer, a personal computer, a mainframe computer, a laptop computer, a tablet computer, a desktop computer, etc.), data stores (e.g., hard disks, memories, databases), networks, software components, and/or hardware components that may be used implement application services for receiving and processing user input.

In an embodiment, the application management system 120 may be communicatively coupled to client devices 102-106 and data repository 130 via network 110. The application management system 120 includes the data communication layer 121, the data management layer 122, the data processing service 123, calculation services 140, analysis models 150, the conversational analysis model 126, the instruction generation service 127, and the interactive exercise generation service 128. Other embodiments of the application management system 120 may include more or less components than depicted in FIG. 5.

In an embodiment, the calculation services 140 may represent a collection of health calculation services implemented to calculate various data health values representing health measurements of the user. For example, the calculation services 140 may include the postural health calculation service 124, which is implemented to calculate postural health values for one or more areas on the user's body based upon the location coordinates of the anatomical landmarks of the user. In an embodiment, the health calculation services 142 represent one of more services implemented to calculate health data values related to various body systems including, but not limited to, vision, the integumentary system, the nervous system, the endocrine system, the lymphatic and immune system, the respiratory system, the digestive system, the urinary system, and the reproductive system.

In an embodiment, the analysis models 150 may represent a collection of machine-learned models each implemented to determine treatment plans for the user based upon health data values calculated by the calculation services 140. For example, the postural health analysis model 125 may be programmed or configured to implement a machine-learned model for selecting one or more treatment plans based on the calculated postural health values. In an embodiment, the health analysis models 152 may represent the implementation of one or more machine-learned models implemented to receive, as input, health data values representing observations related to various body systems including, but not limited to, vision, the integumentary system, the nervous system, the endocrine system, the lymphatic and immune system, the respiratory system, the digestive system, the urinary system, and the reproductive system.

In an embodiment, the health analysis models 152 may generate and train specific machine-learned models directed to generating output that may be used to create one or more treatment plans for therapy and/or one or more medical reports, such as user diagnosis reports, that detail one or more identified ailments associated with the user. The one or more medical reports may then be packaged and sent to one or more external services for further analysis and treatment. For example, the one or more medical reports may represent referrals to external health care professionals. The one or more medical reports may contain, the identified ailments of the user along with observational data that shows why the referral was made so that an external health care professional may treat the user for the identified ailments.

In an embodiment, the health analysis models 152 may implement any machine learning technique to generate the machine-learned models. Examples of machine learning algorithms implemented by the health analysis models 152 may include, but are not limited to, logistic regression, linear regression, random forest, decision tree learning, association rule learning, artificial neural network, support vector machines, and/or Bayesian networks. Embodiments are not limited to any particular type of machine learning technique or algorithm.

In one example the health calculation services 142 and the health analysis models 152 are implemented to identify ailments related to a user's vision. In this example, the system 500 may receive input data from client devices 102-106, where client devices 102-106 may represent camera-based devices implemented to scan the user's eyes, such as a high-resolution video camera or a retinal scanner. The data input may be sent to the application management system 120. The data communication layer 121 may receive the input data and the data processing service 123 may format the input data into a standardized format. The health calculation service 142 may input the standardized input data and calculate one or more health data values associated with evaluating the health of the user's vision. For example, the health data values may represent data points used to determine whether the user is suffering from macular degeneration or any other form of vision impairment. The health data values may then be provided as input into the health analysis model 152. The health analysis model 152 may be configured to compare the health data values to historical health data values of other users who may have been suffering from various vision impairments. The health analysis model 152 may generate one or more user diagnosis reports that may be sent to one or more external health care professionals for follow up treatment. Additionally, depending upon the type and level of vision impairment detected, the health analysis model 152 may generate one or more treatment plans that include eye exercises for the user to perform. The instruction generation service 127 may be used to present the one or more treatment plans to the user.

In another example the health calculation services 142 and the health analysis models 152 are implemented to identify ailments related to a user's integumentary system. In this example, the system 500 may receive input data from client devices 102-106, where client devices 102-106 may represent camera-based devices implemented to detect changes to the user's skin, such as a high-resolution video camera. The data input may be sent to the application management system 120. The data communication layer 121 may receive the input data and the data processing service 123 may format the input data into a standardized format. The health calculation service 142 may input the standardized input data and calculate one or more health data values associated with evaluating the health of the user's skin. For example, the health calculation service 142 may compare input from several different dates in order to determine any changes to the user's skin over time. The calculated health data values may represent data points used to determine whether the user is suffering from a skin condition such as a rash. The health data values may then be provided as input into the health analysis model 152. The health analysis model 152 may be configured to compare the health data values to historical health data values of other users who may have been suffering from various skin issues. The health analysis model 152 may generate one or more user diagnosis reports that may be sent to one or more external health care professionals for follow up treatment, such as sending a referral to a dermatologist. Additionally, depending upon the type and level of skin infection or rash detected, the health analysis model 152 may generate one or more treatment plans that include recommendations for over-the-counter ointments or instructions to observe the skin condition over a period of time and ensure that the condition does not worsen. The instruction generation service 127 may be used to present the one or more treatment plans to the user.

In another example the health calculation services 142 and the health analysis models 152 are implemented to identify ailments related to a user's nervous system. In this example, the system 500 may receive input data from client devices 102-106, where client devices 102-106 may represent a webcam implemented to capture the user-movements, such as when a user is performing exercises presented by the interactive exercise generation service 128 or when prompted to move by the application management system 120. The data input may be sent to the application management system 120. The data communication layer 121 may receive the input data and the data processing service 123 may format the input data into a standardized format. The health calculation service 142 may input the standardized input data and calculate one or more health data values associated with evaluating the user's movements. The calculated health data values may represent data points used to determine whether specific user movements are indicative of possible neurological disorders. The health data values may then be provided as input into the health analysis model 152. The health analysis model 152 may be configured to compare the health data values to historical health data values of other users whose movements may help identify various neurological disorders in the user. The health analysis model 152 may generate one or more user diagnosis reports that may be sent to one or more external health care professionals for follow up treatment, such as sending referral and a report to a neurologist. Additionally, depending upon the type and level of movement disorders detected, the health analysis model 152 may generate one or more treatment plans that include recommendations of exercises to perform prior to seeking treatment from a neurologist. The instruction generation service 127 may be used to present the one or more treatment plans to the user.

Similar to the previous example, the application management system 120 and its health calculation services 142 and health analysis models 152 may be configured to analyze user movements to identify possible medical issues related to the endocrine system. If issues are identified by the health analysis model 152, then the health analysis model 152 may generate one or more user diagnosis reports and/or referrals for an internal medicine consultation.

In another example the health calculation services 142 and the health analysis models 152 are implemented to identify ailments related to a user's cardiovascular system. In this example, the system 500 may receive input data from client devices 102-106, where client devices 102-106 may represent webcams and other devices that may capture a user's heart rate, blood pressure, and any other observational data related to the cardiovascular system. The data input may be sent to the application management system 120. The data communication layer 121 may receive the input data and the data processing service 123 may format the input data into a standardized format. The health calculation service 142 may input the standardized input data and calculate one or more health data values associated with evaluating the user's cardiovascular health. The calculated health data values may represent data points used to determine whether the user is suffering from any possible cardiovascular disorders. The health data values may then be provided as input into the health analysis model 152. The health analysis model 152 may be configured to compare the health data values to historical health data values of other users identified as having cardiovascular disorders. The health analysis model 152 may generate one or more user diagnosis reports that may be sent to one or more external health care professionals for follow up treatment, such as sending referral and a report to a cardiologist. Additionally, the health analysis model 152 may generate one or more treatment plans that include recommendations of exercises to help alleviate any cardiovascular issues the user may be suffering from. The instruction generation service 127 may be used to present the one or more treatment plans to the user.

In another example the health calculation services 142 and the health analysis models 152 are implemented to identify ailments related to a user's lymphatic system. In this example, the system 500 may receive input data from client devices 102-106, where client devices 102-106 may represent webcams and other devices that may capture observations related to the user's immune system. The data input may be sent to the application management system 120. The data communication layer 121 may receive the input data and the data processing service 123 may format the input data into a standardized format. The health calculation service 142 may input the standardized input data and calculate one or more health data values associated with evaluating the user's immune health. The calculated health data values may represent data points used to determine whether the user is suffering from any possible immune disorders. Additionally, the data input may include inputted text or voice from the user. The inputted text or voice may include any subjective information about any health issues the user may be feeling. The health data values may then be provided as input into the health analysis model 152. The health analysis model 152 may be configured to compare the health data values to historical health data values of other users identified as having immune disorders. The health analysis model 152 may generate one or more user diagnosis reports that may be sent to one or more external health care professionals for follow up treatment, such as sending referral and a report to an immunologist.

In another example the health calculation services 142 and the health analysis models 152 are implemented to identify ailments related to a user's respiratory system. In this example, the system 500 may receive input data from client devices 102-106, where client devices 102-106 may represent webcams and other devices that may capture observations related to the user's respiratory system. Other types of client devices may include devices configured to measure your airflow when breathing as well as the level of carbon dioxide expelled when breathing out. The data input may be sent to the application management system 120. The data communication layer 121 may receive the input data and the data processing service 123 may format the input data into a standardized format. The health calculation service 142 may input the standardized input data and calculate one or more health data values associated with evaluating the user's respiratory health. The calculated health data values may represent data points used to determine whether the user is suffering from any possible respiratory disorders. Additionally, the data input may include inputted text or voice from the user. The inputted text or voice may include any subjective information about any breathing issues the user may be feeling. The health data values may then be provided as input into the health analysis model 152. The health analysis model 152 may be configured to compare the health data values to historical health data values of other users identified as having respiratory disorders. The health analysis model 152 may generate one or more user diagnosis reports that may be sent to one or more external health care professionals for follow up treatment.

In another example the health calculation services 142 and the health analysis models 152 are implemented to identify ailments related to a user's digestive system. In this example, the system 500 may receive input data from client devices 102-106, where client devices 102-106 may represent webcams and other devices that may capture observations and movements related to the user's digestive system. For example, the webcams may capture that the user has a distended abdomen. The data input may be sent to the application management system 120. The data communication layer 121 may receive the input data and the data processing service 123 may format the input data into a standardized format. The health calculation service 142 may input the standardized input data and calculate one or more health data values associated with evaluating the user's digestive health. The calculated health data values may represent data points used to determine whether the user is suffering from any possible digestive disorders. Additionally, the data input may include inputted text or voice from the user. The inputted text or voice may include any subjective information about any stomach or intestinal pains the user may be feeling. The health data values may then be provided as input into the health analysis model 152. The health analysis model 152 may be configured to compare the health data values to historical health data values of other users identified as having digestive issues. The health analysis model 152 may generate one or more user diagnosis reports that may be sent to one or more external health care professionals for follow up treatment.

In another example the health calculation services 142 and the health analysis models 152 are implemented to identify ailments related to a user's urinary system. In this example, the system 500 may receive input data from client devices 102-106, where client devices 102-106 may represent webcams and other devices that may capture observations and movements related to the user's urinary system. For example, the webcams may capture that the user constantly doubles over or pauses the video session to go to the bathroom. The data input may be sent to the application management system 120. The data communication layer 121 may receive the input data and the data processing service 123 may format the input data into a standardized format. The health calculation service 142 may input the standardized input data and calculate one or more health data values associated with evaluating the user's urinary health. The calculated health data values may represent data points used to determine whether the user is suffering from any possible urinary disorders, such as a urinary tract infection. Additionally, the data input may include inputted text or voice from the user. The inputted text or voice may include any subjective information about any intestinal pains or bladder pains the user may be feeling. The health data values may then be provided as input into the health analysis model 152. The health analysis model 152 may be configured to compare the health data values to historical health data values of other users identified as having urinary issues. The health analysis model 152 may generate one or more user diagnosis reports that may be sent to one or more external health care professionals for follow up treatment.

In another example the health calculation services 142 and the health analysis models 152 are implemented to identify ailments related to a user's reproductive system. In this example, the system 500 may receive input data from client devices 102-106, where client devices 102-106 may represent webcams and other devices that may capture observations and movements related to the user's reproductive system. The data input may be sent to the application management system 120. The data communication layer 121 may receive the input data and the data processing service 123 may format the input data into a standardized format. The health calculation service 142 may input the standardized input data and calculate one or more health data values associated with evaluating the user's reproductive health. The calculated health data values may represent data points used to determine whether the user is suffering from any possible reproductive disorders. Additionally, the data input may include inputted text or voice from the user. The inputted text or voice may include any subjective information about any pains, related to the reproductive system, the user may be feeling. The health data values may then be provided as input into the health analysis model 152. The health analysis model 152 may be configured to compare the health data values to historical health data values of other users identified as having digestive issues. The health analysis model 152 may generate one or more user diagnosis reports that may be sent to one or more external health care professionals for follow up treatment.

In an embodiment, each of the above examples implementing the health calculation services 142 and the health analysis models 152 may include subjective data input provided by the user. For example, the user may use a chat box to convey subjective information related to symptoms the user may be experiencing. The conversational analysis model 126 may be implemented to analyze the subjective input and provide relevant health data values representing the subjective input to the health analysis models 152 for additional analysis.

### Processing Overview

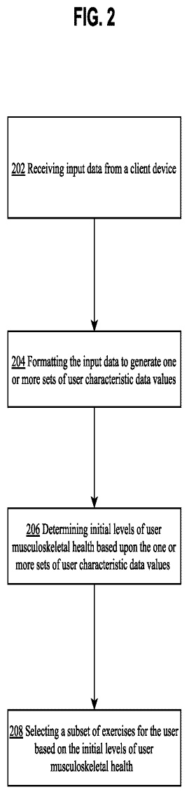

FIG. 2 depicts an example flowchart for analyzing user input data, determining levels of musculoskeletal health for the user, and selecting a set of treatment plans based on the user's musculoskeletal health, in accordance with an embodiment. Process 200 may be performed by a single program or multiple programs. The operations of the process as shown in FIG. 2 may be implemented using processor-executable instructions that are stored in computer memory. For purposes of providing a clear example, the operations of FIG. 2 are described as performed by components of system 100. For the purposes of clarity process 200 is described in terms of a single entity.

In operation 202, process 200 receives input data from a client device. In an embodiment, the data communication layer 121 may receive from client device 102, input data from a user. Input data may represent any form of captured images, video, audio, text, and file input from a user. For example, client device 102 may represent a webcam enabled computer that captures input data as a series of video frames of the user standing upright. Client device 102 may send the series of video frames of the user standing upright to the data communication layer 121.

In operation 204, process 200 formats the input data to generate one or more sets of user measurements. In an embodiment, the data processing service 123 receives the input data and formats the input data to generate the one or more sets of user measurements. For example, if the user input represents a digital image or a series of video frames depicting the user standing upright and facing client device 102, then the data processing service 123 may analyze the received image or video frames to identify anatomical landmarks of the user. The data processing service 123 may assign spatial coordinates to each of the identified anatomical landmarks. The anatomical landmarks and their spatial coordinates may represent a set of user measurements. In another example, the data processing service 123 may receive an audio recording of the user responding to a series of questions prompted by the application management system 120. The data processing service 123 may process the audio recording to identify keywords and phrases in user responses and generate data objects that contain the question/prompt and the corresponding user response. In yet other examples, the data processing service 123 may format various types of user input to generate sets of user characteristic data values that may be used to determine the user's musculoskeletal health.

In operation 206, process 200 determines initial levels of user musculoskeletal health based upon the one or more sets of user measurements. In an embodiment, the postural health calculation service 124 calculates postural health values for the user based upon the one or more sets of user measurements. For example, when the one or more sets of user measurements includes anatomical landmarks and their corresponding spatial coordinates, the postural health calculation service 124 calculates postural health values for one or more areas on the user's body based upon the location coordinates of the anatomical landmarks of the user. Units for the postural health values may be based on a 0-100% scale or any other scaling system configured by the application management system 120. Further details describing calculating a user's musculoskeletal health are described in the POSTURAL HEALTH CALCULATION section herein.

In operation 208, process 200 selects a subset of exercises for the user based on the initial levels of user musculoskeletal health of the user. In an embodiment, the postural health analysis model 125 may implement a machine-learned model configured to select a subset of exercises for the user based upon the initial levels of user musculoskeletal health calculated by the postural health calculation service 124. The postural health analysis model 125 may also receive, as input, user characteristic data values representing audio speech and/or text speech. In an embodiment, the conversational analysis model 126 may parse and identify relevant user responses to determine whether the user is experiencing pain in a particular area or during particular movements. The postural health analysis model 125 may use the relevant user responses along with the initial levels of user musculoskeletal health to select specific treatment plans that emphasize a subset of exercises designed for the user.

In an embodiment, the selected subset of exercises for the user may be presented to the user in various formats including, but not limited to, video instruction, interactive video instruction, written instructions, audio instructions, and any other media format. The instruction generation service 127 may send to client device 102 instructions for the selected subset of exercises. For example, the instruction generation service 127 may render a graphical user interface for display and/or playing instructional exercise videos to the user. Alternatively, the interactive exercise generation service 128 may present a graphical user interface configured to play an interactive exercise video, which superimposes a video representation of the user, in real-time, onto the interactive video. Video representations of the user may include live video, an avatar, and any other graphical representation of a human.

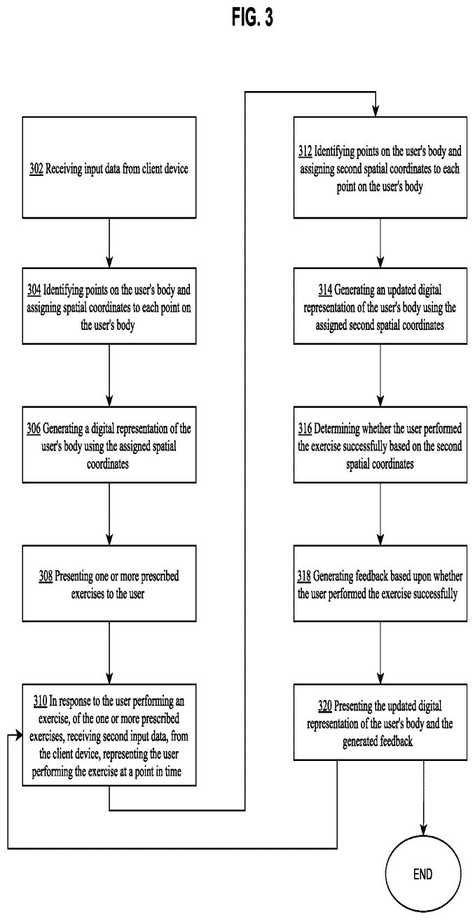

FIG. 3 depicts an example flowchart for displaying an interactive video that contains a digital representation of the user performing the exercises, in accordance with an embodiment. Process 300 may be performed by a single program or multiple programs. The operations of the process as shown in FIG. 3 may be implemented using processor-executable instructions that are stored in computer memory. For purposes of providing a clear example, the operations of FIG. 3 are described as performed by components of system 100. For the purposes of clarity process 300 is described in terms of a single entity.

In operation 302, process 300 receives input data from a client device. In an embodiment, the data communication layer 121 may receive from client device 102, input data from a user. Input data may represent any form of captured images, video, audio, text, and file input from a user. For example, the input data may be a captured image of the user standing.

In operation 304, process 300 identifies points on the user's body and assigns spatial coordinates to each point on the user's body. In an embodiment, the data processing service 123 may assign spatial coordinates to each of the identified anatomical landmarks of the user. These coordinates may be compared to the initial coordinates measured during the initial user assessment and may be used as a baseline measurement for interactive video exercises.

In operation 306, process 300 generates a digital representation of the user's body using the assigned spatial coordinates. In an embodiment, the interactive exercise generation service 128 generates a digital representation of the user. The digital representation may be cropped from a live video feed captured by client device 102. Alternatively, the digital representation of the user may be an avatar, stick figure, block figure, or cartoon character. The digital representation of the user may be used within the interactive exercise video presented by the interactive exercise generation service 128.

In operation 308, process 300 presents the one or more prescribed exercises to the user. In an embodiment, the interactive exercise generation service 128 generates an interactive graphical user interface and sends the data for the interactive graphical user interface to client device 102 for presentation to the user.

In operation 310, in response to the user performing the presented exercise, process 300 receives second input data, from client device 102, representing the user performing the exercise at a point in time. In an embodiment, the data communication layer 121 receives from client device 102 the second input data. For example, if the presented exercise is the performance of a squat exercise, then the second input data may be an image of the user performing the squat exercise.

In operation 312, process 300 identifies points on the user's body and assigns second spatial coordinates to each point on the user's body. In an embodiment, the data processing service 123 may assign second spatial coordinates to each of the identified anatomical landmarks of the user. For instance, the second spatial coordinates for each anatomical landmark may represent the positions that each anatomical landmark is in during performance of the squat exercise at a specific point in time.

In operation 314, process 300 generates an updated digital representation of the user's body using the assigned second spatial coordinates. In an embodiment, the interactive exercise generation service 128 generates the updated digital representation of the user while performing the squat exercise. The updated digital representation may be displayed in the interactive exercise video so that the user can see himself performing the exercise in real-time or near real-time.

In operation 316, process 300 determines whether the user performed the exercise successfully based on the second spatial coordinates. In an embodiment, the interactive exercise generation service 128 may compare the second spatial coordinates, representing the identified anatomical landmarks of the user during the squat exercise, to target spatial coordinates that represent where the user's anatomical landmarks should be when performing the squat exercise correctly.

In operation 318, process 300 generates feedback based upon whether the user performed the exercise successfully. In an embodiment, the interactive exercise generation service 128 may send positive or negative feedback to the user depending upon whether the user performed the exercise successfully. For instance, the interactive exercise generation service 128 may superimpose a thumbs up or any other graphical image or icon onto the interactive video in order to indicate that the user successfully performed the exercise. If the user did not successfully perform the exercise, then the interactive exercise generation service 128 may superimpose a thumbs down or any graphical image or graphical icon that represents negative feedback indicating that the user did not perform the exercise successfully.

In another embodiment, the interactive exercise generation service 128 may send an audible sound or haptic feedback to the user to indicate that the user either successfully or unsuccessfully performed the exercise. For example, the haptic feedback or audible sound may be sent to another client device 104, such as a smartwatch or wireless headphones. In yet another embodiment, the interactive exercise generation service 128 may superimpose a graphical image onto the interactive video displayed on client device 102, simultaneously send haptic feedback to client device 104, and/or send an audible sound to either client device 102 or client device 104 to indicate whether the user performed the exercise successfully.

In operation 320, process 300 presents the updated digital representation of the user's body and the generated feedback to the user. In an embodiment, the interactive exercise generation service 128 sends the interactive video to client device 102 that contains an updated digital representation of the user's body and the generated feedback.

In an embodiment, process 300 may return to operation 310 if the user is performing additional exercises prescribed or has not yet finished the previously prescribed exercise. For example, if the squat exercise is to be performed 10 times, then process 300 would proceed back to operation 310 to receive additional input data representing a new position of the user while performing the prescribed exercise.

### Postural Health Calculation

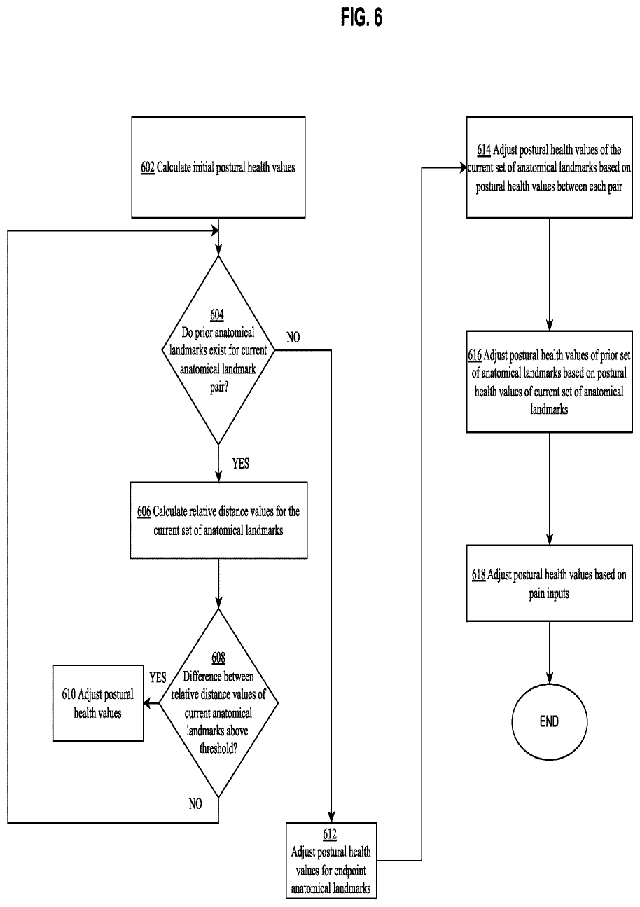

In an embodiment, postural health values for different regions of the body are calculated based upon relative locations of anatomical landmarks of a user. For example, a postural health value for a user's right shoulder region may be calculated based on spatial coordinates of an anatomical landmark representing the user's right shoulder and relative distance to other anatomical landmarks of the user near the user's right shoulder. FIG. 6 depicts an example flowchart for calculating postural health values for anatomical landmarks identified for a user, in accordance with an embodiment. Process 600 may be performed by a single program or multiple programs. The operations of the process as shown in FIG. 6 may be implemented using processor-executable instructions that are stored in computer memory. For purposes of providing a clear example, the operations of FIG. 6 are described as performed by components of system 100. For the purposes of clarity process 600 is described in terms of a single entity.

In an embodiment, the postural health calculation service 124 is configured to calculate postural health values corresponding to each of the anatomical landmarks identified from a user's image. The user's image may be from a digital image of the user or a video frame of the user. The data processing service 123 provides the postural health calculation service 124 with a set of anatomical landmarks that include spatial coordinates for each anatomical landmark.

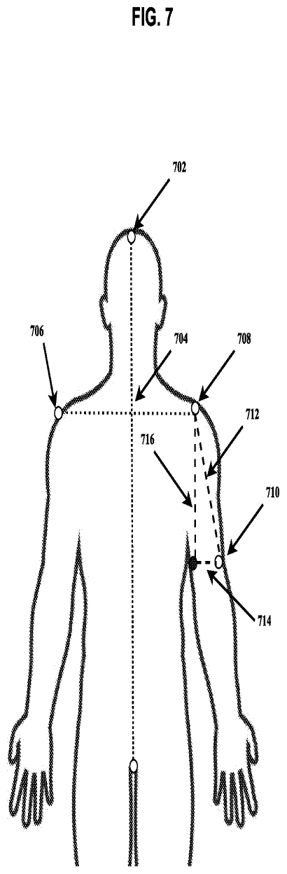

In operation 602, process 600 calculates initial postural health values for the set of anatomical landmarks. In an embodiment, the postural health calculation service 124 identifies a middle x-axis position that represents the middle point of the user's body. The middle x-axis position may be based on median x-axis values between left and right anatomical landmarks. FIG. 7 depicts an example image of a user's body and example anatomical landmarks used to determine postural health values for the user, according to an embodiment. Middle x-axis 702 represents an identified middle x-axis position.

In an embodiment, the postural health calculation service 124 calculates a left deviation and right deviation. The left deviation represents the horizontal distance between a left anatomical landmark and the middle x-axis position. The right deviation represents the horizontal distance between a right anatomical landmark and the middle x-axis position. The left and right deviations are calculated for each set of left and right anatomical landmarks. For example, referring to FIG. 7, left anatomical landmark (LAL) 706 represents the left anatomical landmark for the left shoulder. The left deviation for LAL 706 is calculated as the horizontal distance between LAL 706 and the middle point 704, which is a point on middle x-axis 702. Right anatomical landmark (RAL) 708 represents the right anatomical landmark for the right shoulder. The right deviation for RAL 708 is calculated as the horizontal distance between RAL 708 and the middle point 704. In an embodiment, left and right deviations are calculated for each of the sets of anatomical landmarks.

In an embodiment, the postural health calculation service 124 calculates a deviation scaling factor that is used to convert left and right deviation values into a scaled deviation value. For example, the deviation scaling factor may be calculated as:

$$d S = \frac{2}{( l D + r D )}$$

where dS is the deviation scaling factor, lD is the left deviation, and rD is the right deviation.

In an embodiment, the postural health calculation service 124 calculates initial postural health values by multiplying the left and right deviation values by the deviation scaling factor and rounding down the result to the nearest whole value. The following equations represent one example for calculating the initial postural health values for one set of anatomical landmarks as:

$$lE = floor ( lD * dS )$$

$$rE = floor ( rD * dS )$$

where lE is the left postural health value and rE is the right postural health value. In another example, the left and right postural health values may be calculated differently, such as multiplying the left and right deviation values by the deviation scaling factor and rounding up to the nearest whole value. In yet other examples, the postural health values may be based on multiplying the left and right deviation values by the deviation scaling factor without rounding the results.

In operation 604, process 600 determines whether prior anatomical landmarks exist for a current set of anatomical landmarks. Prior anatomical landmarks represent the anatomical landmark that is directly below the current anatomical landmark. For example, if the current anatomical landmark is the left shoulder, then the prior anatomical landmark would be the landmark that represents the left elbow point. Referring to FIG. 7 and RAL 708, where RAL 708 represents the current anatomical landmark, then the prior anatomical landmark would be prior right anatomical landmark (pRAL) 710. If at operation 604, process 600 determines that there are prior anatomical landmarks for the set of current anatomical landmarks, then process 600 proceeds to operation 606 to determine relative distance values between the set of current anatomical landmarks and the prior anatomical landmarks. If, however, at operation 604 process 600 determines that there are not prior anatomical landmarks, then process 600 proceeds to operation 612. In an embodiment, process 600 may determine that there are no prior anatomical landmarks when the current set of anatomical landmarks represents the left and right heels. In another embodiment, process 600 may skip to operation 612 when dealing with specific anatomical landmarks, such as landmarks representing head points (car, jaw, eye, and chin). Anatomical landmarks associated with the head may be treated differently as these landmarks may be directly influenced by the shoulder anatomical landmarks as opposed to a directly adjacent anatomical landmark.

Referring back to operation 606, process 600 calculates relative distance values for the current set of anatomical landmarks. In an embodiment, the postural health calculation service 124 calculates relative distance for the current set of anatomical landmarks by calculating the spatial distances between the left and right anatomical landmarks of the current set and the left right anatomical landmarks of the prior set of anatomical landmarks. In one example, relative distance values are calculated as the square root of the sum of square differences of the x and y values between the current anatomical landmark and the prior anatomical landmark. The following equations represent calculating the relative distance values as:

$$l H = hypot ( L A H ( x ) - p L A H ( x ) , LA H ( y ) - p L A H ( y ) )$$

$$rH = hypot ( R A H ( x ) - p R A H ( x ) , RA H ( y ) - p R A H ( y ) )$$

where lH is the left relative distance between the current left anatomical landmark and the prior left anatomical landmark, and rH is the right relative distance between the current right anatomical landmark and the prior right anatomical landmark. The hypot function represents a hypotenuse function for calculating the relative distance between two points using the differences between x and y spatial coordinates. The input for the hypot function is the x-axis difference between x-spatial coordinates LAH(x)−pLAH(x) and the y-axis difference between y-spatial coordinates LAH(y)−pLAH(y). In FIG. 7, the x-axis difference between the spatial coordinates of RAL 708 and pRAL 710 is represented by line 714 and the y-axis difference is represented by line 716. Relative distance line 712 represents the right relative distance value rH for the right anatomical landmark.

In operation 608, process 600 determines whether the difference between the left relative distance and the right relative distance is above a particular threshold. The particular threshold may represent a threshold value that ensures that the relative distance values are within an acceptable range. If the relative distance of one side of an anatomical landmark is much larger than the relative distance for the opposite side of an anatomical landmark, then the postural health value for the anatomical landmark with the lower relative distance is increased to compensate for the difference. In an embodiment, the particular threshold may be based on a percentage value, such as 10%, or may be based on a static value such as centimeters, millimeters, inches, or any other value. For example, if the left relative distance is greater than 10% higher than the right relative distance, then the postural health value of the right postural health value may be adjusted to compensate for the disparity in distance values. If the difference between the relative distance values is greater than the particular threshold, then process 600 proceeds to operation 610. If, however, the difference between the relative distance values is less than or equal to the particular threshold, then process 600 proceeds back to operation 604 to process the next set of anatomical landmarks.

At operation 610, process 600 adjusts a postural health value for the current set of anatomical landmarks. In an embodiment, postural health calculation service 124 may increment the postural health value of one of the anatomical landmarks based on which landmark (left or right anatomical landmark) had a smaller relative distance value. For example, if the particular threshold is set to 10% and the relative distance value for LAL 706 is 10 and the relative distance value for RAL 708 is 7, then the postural health value for RAL 708 may be increased by one point. The increase in of the postural health value for RAL 708 may represent a decrease in the observed postural health related to RAL 708 (right shoulder).

At operation 612, process 600 adjusts postural health values for endpoint anatomical landmarks. In an embodiment, postural health calculation service 124 may adjust postural health values associated with specific landmarks, such as endpoint landmarks. Endpoint landmarks may represent landmarks such as foot or heel landmarks that have no other prior landmarks. The postural health calculation service 124 determines a current x-axis middle point and a previous x-axis middle point. The current x-axis middle point represents a point between the current left anatomical landmark and the right anatomical landmark. The previous x-axis middle point represents a point between the previous left anatomical landmark and the previous right anatomical landmark. In the example where the current landmarks represent the heel, then the previous landmarks would represent the foot. After determining the current x-axis middle point and the previous x-axis middle point, the postural health calculation service 124 determines whether the difference between the current and previous x-axis middle point is above a specific threshold. The specific threshold may be a configurable value that is either based on a percentage difference or a static value. For instance, the specific threshold may be configured to 5%. In other examples the specific threshold may be higher or lower. For example, if the specific threshold is set to 5% and the current x-axis middle point value is 10 and the previous x-axis middle point value is 9, then the postural health value for LAL 706 may be increased by one point. If, however, the current x-axis middle point value is 10 and the previous x-axis middle point value is 11, then the postural health value for RAL 708 may be increased by one point.

In operation 614, process 600 adjusts postural health values of the current set of anatomical landmarks based on postural health values between each pair. In an embodiment, if the postural health value of one side of the current set of anatomical landmarks is significantly greater than the other side of the current set, then the postural health calculation service 124 may adjust the postural health values for the current set of anatomical landmarks. In one example, if the postural health value of the current left anatomical landmark is more than double the postural health value of the current right anatomical landmark, then the postural health value of the current right anatomical landmark may be set to ½ of the value of the current left anatomical landmark. The following equation represents one embodiment for adjusting the postural health value of the right anatomical landmark using horizontal normalization:

$$If [ lE \geq 2 * rE ] , then rE = round ( \frac{l E}{2} )$$

where lE is the postural health value for the left anatomical landmark and rE is the postural health value for the right anatomical landmark, such that if lE is greater than or equal to twice the value of rE, then the value of rE is adjusted to equal ½ of the value of lE rounded to the nearest whole value.

Similarly, the following equation represents one embodiment for adjusting the postural health value of the left anatomical landmark using horizontal normalization:

$$If [ rE \geq 2 * lE ] , then lE = round ( \frac{rE}{2} )$$

where if rE is greater than or equal to twice the value of lE, then the value of lE is adjusted to equal ½ of the value of rE rounded to the nearest whole value. In other embodiments, the rE and lE may be adjusted by a different amount.

In operation 616, process 600 adjusts postural health values of the prior set of anatomical landmarks based on postural health values of the current set of anatomical landmarks. In an embodiment, if there is a large discrepancy between postural health values of the prior set of anatomical landmarks and the current set of anatomical landmarks, then the postural health calculation service 124 may adjust the postural health values for the prior set of anatomical landmarks. The purpose for doing this adjustment is to ensure that anatomical landmarks that are adjacent to each other do not have large discrepancies between postural health values. That is, anatomical regions that are next to each other, such as the shoulder and elbow or hip and knee, are likely to affect each other and should not have large discrepancies between their postural health values. In one example, if the postural health value of the current left anatomical landmark is more than double the postural health value of the prior left anatomical landmark, then the postural health value of the prior left anatomical landmark may be set to ½ of the value of the current left anatomical landmark. The following equation represents one embodiment for adjusting the postural health value of the prior left anatomical landmark using vertical normalization:

$$If [ lE \geq 2 * plE ] , then plE = round ( \frac{lE}{2} )$$

where lE is the postural health value for the current left anatomical landmark and plE is the postural health value for the prior left anatomical landmark, such that if lE is greater than or equal to twice the value of plE, then the value of plE is adjusted to equal ½ of the value of lE rounded to the nearest whole value.

Similarly, the following equation represents one embodiment for adjusting the postural health value of the prior right anatomical landmark using horizontal normalization:

$$If [ rE \geq 2 * prE ] , then prE = round ( \frac{rE}{2} )$$

where if rE (current right) is greater than or equal to twice the value of prE (prior right), then the value of prE is adjusted to equal ½ of the value of rE rounded to the nearest whole value. In other embodiments, the rE and lE may be adjusted by a different amount.

In operation 618, process 600 adjusts postural health values based on pain inputs provided by the user. In an embodiment, the data processing service 123 may receive from client device 102 user input that specifies pain input for regions of the body represented by one or more anatomical landmarks. The pain input may include, but is not limited to, a pain intensity value and a pain frequency value, where the pain intensity value represents how intense the pain is at a particular anatomical landmark and the pain frequency value represents how frequent the pain is observed at the particular anatomical landmark. For example, if a user has very intense pain in his right knee, then the pain intensity may be a 10 out of 10. If the user's right knee pain is infrequent then the pain frequency may be a 1 out of 100.

In an embodiment, the postural health calculation service 124 may adjust the postural health values for sets of anatomical landmarks based on pain inputs for each anatomical landmark. The amount adjusted to the postural health value of a particular anatomical landmark may be inversely proportional to the current postural health value. For example, if the current postural health value is a low value, such as 0 or 1 (out of a 4-point scale), then the pain input may have greater weight to adjusting the current postural health value. Conversely, if the current postural health value is a high value, such as 3 or 4 (out of a 4-point scale), then the pain input may have lesser weight to adjusting the current postural health value. The following equation represents one example of adjusting the postural health value for a left anatomical landmark based on the pain inputs of intensity and frequency:

$$l E = l E + ( 4 - l E ) * ( \frac{l f r e q}{10} ) * ( \frac{l i n t}{1000} )$$

where lfreq is the left anatomical landmark pain frequency (measured out of a 10-point scale), lint is the left anatomical landmark pain intensity (measured out of a 1000-point scale), (4−lE) represents the inverse of the current postural health value where the postural health value scale is measured from 0 to 4.

For example, if a user's left postural health value for his left knee equals lE=2 and his pain frequency is 5 out of 10 and his pain intensity is 100 out of 1000, then the adjusted postural health value would be:

$$l E = {2}_{l E} + ( 4 - {2}_{l E} ) * ( \frac{{5}_{l f r e q}}{10} ) * ( \frac{10 {0}_{lint}}{1000} )$$

$$lE = {2}_{l E} + 0.1 = 2.1$$

where the adjusted postural health value for the left knee, accounting for pain intensity and frequency equals 2.1.

Similarly, the following equation represents one example of adjusting the postural health value for a right anatomical landmark based on the pain inputs of intensity and frequency:

$$r E = r E + ( 4 - r E ) * ( \frac{r f r e q}{10} ) * ( \frac{r i n t}{1000} )$$

where rfreq is the right anatomical landmark pain frequency (measured out of a 10-point scale), rint is the right anatomical landmark pain intensity (measured out of a 1000-point scale), (4−rE) represents the inverse of the current postural health value where the postural health value scale is measured from 0 to 4. In other examples, the postural health values may be adjusted using different postural health value ranges, different pain intensity ranges, and different pain frequency ranges.

In an embodiment, postural health calculation service 124 may adjust the postural health values for specific sets of anatomical landmarks that may be related to areas of the body that greatly affect each other. For example, hip pain may be closely related to lower back. For this reason, the postural health calculation service 124 may select the dominant pain from either the lower back or hip when adjusting postural health values for both the hip and lower back anatomical landmarks. The following equations represent applying the dominant pain to the postural health values for the hip and lower back:

$$lfreq , lint = max Pain ( leftLowerback , Lefthip )$$

$$rfreq , rint = max Pain ( rightLowerBack , rightHip )$$

$$lE = lE + ( 4 - l E ) * ( \frac{l f r e q}{10} ) * ( \frac{l i n t}{1000} )$$

$$rE = rE + ( 4 - r E ) * ( \frac{r f r e q}{10} ) * ( \frac{r i n t}{1000} )$$

where maxPain(leftLowerBack, LeftHip) represents a function to select the pain frequency and intensity from the region that is more intense, either the left lower back or left hip region, and maxPain(rightLowerBack, rightHip) represents a function to select the pain frequency and intensity from the region that is more intense, either the right lower back or right hip region. Embodiments for selecting a dominant pain region are not limited to only the lower back and hip. The maxPain function may be applied to any group of anatomical landmarks.

In an embodiment, postural health calculation service 124 may adjust the postural health values for specific sets of anatomical landmarks that may be related to areas of the body that may have more intense pain, such as the head and neck region. For example, pain inputs related to the head and/or neck region may boosted as:

$$lfreq = left neck pain frequency * 1.5$$

$$lint = left neck pain intensity * 1.5$$

$$rfreq = right neck pain frequency * 1.5$$

$$rint = right neck pain intensity * 1.5$$

$$lE = l E + ( 4 - l E ) * ( \frac{l f r e q}{10} ) * ( \frac{l i n t}{1000} )$$

$$rE = r E + ( 4 - r E ) * ( \frac{r f r e q}{10} ) * ( \frac{r i n t}{1000} )$$

Embodiments for boosting pain frequencies and intensities are not limited to only the head and neck region and may be applied to any group of anatomical landmarks.

In an embodiment, the adjusted postural health values calculated by the postural health calculation service 124 may be provided to the postural health analysis model 125 to select a subset of exercises.

### Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, portable computer systems, handheld devices, networking devices or any other device that incorporates hard-wired and/or program logic to implement the techniques.

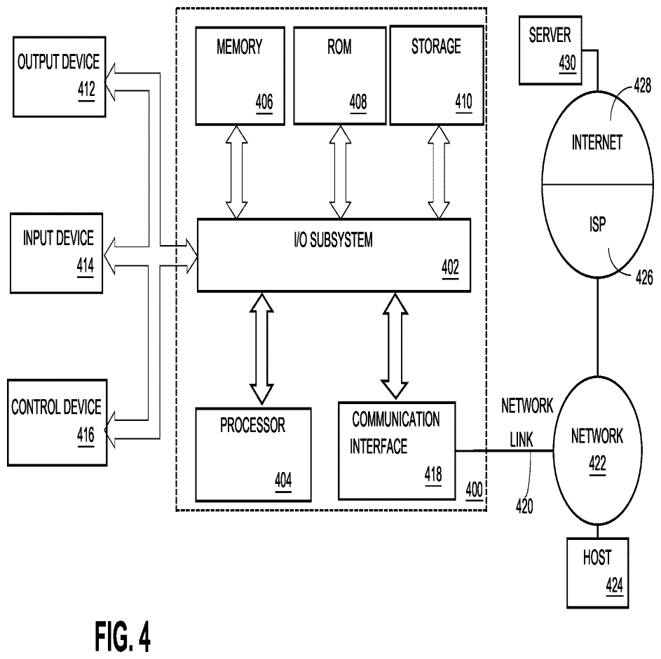

For example, FIG. 4 is a block diagram that illustrates a computer system 400 upon which an embodiment of the invention may be implemented. Computer system 400 includes a bus 402 or other communication mechanism for communicating information, and a hardware processor 404 coupled with bus 402 for processing information. Hardware processor 404 may be, for example, a general-purpose microprocessor.

Computer system 400 also includes a main memory 406, such as a random-access memory (RAM) or other dynamic storage device, coupled to bus 402 for storing information and instructions to be executed by processor 404. Main memory 406 also may be used for storing temporary variables or other intermediate information during execution of instructions to be executed by processor 404. Such instructions, when stored in non-transitory storage media accessible to processor 404, render computer system 400 into a special-purpose machine that is customized to perform the operations specified in the instructions.

Computer system 400 further includes a read only memory (ROM) 408 or other static storage device coupled to bus 402 for storing static information and instructions for processor 404. A storage device 410, such as a magnetic disk, optical disk, or solid-state drive is provided and coupled to bus 402 for storing information and instructions.

Computer system 400 may be coupled via bus 402 to an output device 412, such as a light-emitting diode (LED) display, for displaying information to a computer user. An input device 414, including alphanumeric and other keys, is coupled to bus 402 for communicating information and command selections to processor 404. Another type of user input device is control device 416, such as a mouse, a trackball, or cursor direction keys for communicating direction information and command selections to processor 404 and for controlling cursor movement on the output device 412. This input device typically has two degrees of freedom in two axes, a first axis (e.g., x) and a second axis (e.g., y), that allows the device to specify positions in a plane.

Computer system 400 may implement the techniques described herein using customized hard-wired logic, one or more ASICs or FPGAs, firmware and/or program logic which in combination with the computer system causes or programs computer system 400 to be a special-purpose machine. According to one embodiment, the techniques herein are performed by computer system 400 in response to processor 404 executing one or more sequences of one or more instructions contained in main memory 406. Such instructions may be read into main memory 406 from another storage medium, such as storage device 410. Execution of the sequences of instructions contained in main memory 406 causes processor 404 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical disks, magnetic disks, or solid-state drives, such as storage device 410. Volatile media includes dynamic memory, such as main memory 406. Common forms of storage media include, for example, a floppy disk, a flexible disk, hard disk, solid-state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge.

Storage media is distinct from but may be used in conjunction with transmission media. Transmission media participates in transferring information between storage media. For example, transmission media includes coaxial cables, copper wire and fiber optics, including the wires that comprise bus 402. Transmission media can also take the form of acoustic or light waves, such as those generated during radio-wave and infra-red data communications.

Various forms of media may be involved in carrying one or more sequences of one or more instructions to processor 404 for execution. For example, the instructions may initially be carried on a magnetic disk or solid-state drive of a remote computer. The remote computer can load the instructions into its dynamic memory and send the instructions over a telephone line using a modem. A modem local to computer system 400 can receive the data on the telephone line and use an infra-red transmitter to convert the data to an infra-red signal. An infra-red detector can receive the data carried in the infra-red signal and appropriate circuitry can place the data on bus 402. Bus 402 carries the data to main memory 406, from which processor 404 retrieves and executes the instructions. The instructions received by main memory 406 may optionally be stored on storage device 410 either before or after execution by processor 404.

Computer system 400 also includes a communication interface 418 coupled to bus 402. Communication interface 418 provides a two-way data communication coupling to a network link 420 that is connected to a local network 422. For example, communication interface 418 may be an integrated services digital network (ISDN) card, cable modem, satellite modem, or a modem to provide a data communication connection to a corresponding type of telephone line. As another example, communication interface 418 may be a local area network (LAN) card to provide a data communication connection to a compatible LAN. Wireless links may also be implemented. In any such implementation, communication interface 418 sends and receives electrical, electromagnetic or optical signals that carry digital data streams representing various types of information.

Network link 420 typically provides data communication through one or more networks to other data devices. For example, network link 420 may provide a connection through local network 422 to a host computer 424 or to data equipment operated by an Internet Service Provider (ISP) 426. ISP 426 in turn provides data communication services through the worldwide packet data communication network now commonly referred to as the “Internet” 428. Local network 422 and Internet 428 both use electrical, electromagnetic or optical signals that carry digital data streams. The signals through the various networks and the signals on network link 420 and through communication interface 418, which carry the digital data to and from computer system 400, are example forms of transmission media.

Computer system 400 can send messages and receive data, including program code, through the network(s), network link 420 and communication interface 418. In the Internet example, a server 430 might transmit a requested code for an application program through Internet 428, ISP 426, local network 422 and communication interface 418.

The received code may be executed by processor 404 as it is received, and/or stored in storage device 410, or other non-volatile storage for later execution.

In the foregoing specification, embodiments of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

1. A method comprising:
receiving an image of a user at a musculoskeletal health evaluation system;
processing, by execution of one or more hardware processors of the musculoskeletal health evaluation system, the image by determining a first set of coordinates on the image associated with a first set of anatomical landmarks detected on a left side of the user and a second set of coordinates on the image associated with a second set of anatomical landmarks detected on a right side of the user;
measuring, by execution of the one or more hardware processors of the musculoskeletal health evaluation system, asymmetry at corresponding regions of the left side and the right side of the user, wherein measuring the asymmetry comprises determining that the first set of coordinates for one or more anatomical landmarks from the first set of anatomical landmarks on the left side of the user deviate from the second set of coordinates for one or more corresponding anatomical landmarks from the second set of anatomical landmarks on the right side of the user; and
presenting a graphical user interface with an evaluation of musculoskeletal health based on the asymmetry that is measured at the corresponding regions of the user by the musculoskeletal health evaluation system.

2. The method of claim 1, wherein presenting the graphical user interface comprises:
generating a digital representation of the user based on the first set of coordinates associated with the first set of anatomical landmarks and the second set of coordinates associated with the second set of anatomical landmarks.

3. The method of claim 2, wherein presenting the graphical user interface comprises:
updating the digital representation in response to the asymmetry that is measured at the corresponding regions.

4. The method of claim 3, wherein updating the digital representation comprises:
visually identifying the corresponding regions with the asymmetry on the digital representation.

5. The method of claim 3, wherein updating the digital representation comprises:
visually differentiating the corresponding regions on the digital representation based on the asymmetry that is measured for each of the corresponding regions.

6. The method of claim 1, wherein presenting the graphical user interface comprises:
providing visual feedback as to the musculoskeletal health at the corresponding regions based on the asymmetry that is measured for each of the corresponding regions.

7. The method of claim 1 further comprising:
determining a middle x-axis of the user in the image;
calculating a first amount by which coordinates for a particular landmark on the left side of the user deviate from the middle x-axis and a second amount by which coordinates for the particular landmark on the right side of the user deviate from the middle x-axis; and
wherein measuring the asymmetry comprises calculating a difference between the first amount and the second amount.

8. The method of claim 1 further comprising:
receiving pain inputs representing pain at the corresponding regions; and
generating postural health values for the corresponding regions by calculating the postural health values based on the asymmetry that is measured at each of the corresponding regions and adjusting the postural health values based on the pain inputs.

9. The method of claim 1 further comprising:
receiving a plurality of images of the user at different times;
tracking changes to the asymmetry at the corresponding regions based on changing positioning of the first set of anatomical landmarks and the second set of anatomical landmarks across the plurality of images; and
updating the evaluation of the musculoskeletal health based on the changes to the asymmetry.

10. The method of claim 9, wherein updating the evaluation comprises:
adjusting a treatment plan specifying different exercises for improving the musculoskeletal health based on said tracking of the changes to the asymmetry.

11. The method of claim 1, wherein generating the graphical user interface comprises:
generating a referral to a health care professional based on the asymmetry that is measured at the corresponding regions; and
presenting postural health values that are derived from the asymmetry to the health care professional.

12. The method of claim 1 further comprising:
mapping the asymmetry at the corresponding regions to one or more ailments affecting the corresponding regions; and
wherein generating the graphical user interface comprises presenting the one or more ailments requiring treatment in the graphical user interface.

13. The method of claim 1 further comprising:
determining that a first anatomical landmark from the first set of anatomical landmarks affects postural analysis of a second anatomical landmark from the first set of anatomical landmarks; and
wherein measuring the asymmetry comprises:
determining a first relative distance between the first anatomical landmark and the second anatomical landmark;
determining a second relative distance between a third anatomical landmark of the second set of anatomical landmarks about the right side of the user that corresponds to the first anatomical landmark about the left side of the user and a fourth anatomical landmark of the second set of anatomical landmarks about the right side of the user that corresponds to the second anatomical landmark about the left side of the user; and
calculating a postural value based on a difference between the first relative distance and the second relative distance.

14. The method of claim 13 further comprising:
determining that a fifth anatomical landmark from the first set of anatomical landmarks is not affected by another anatomical landmark from the first set of anatomical landmarks; and
wherein measuring the asymmetry comprises:
determining a third relative distance between the fifth anatomical landmark and a middle point about the user and a fourth relative distance between a sixth anatomical landmark of the second set of anatomical landmarks about the right side of the user that corresponds to the fifth anatomical landmark about the left side of the user and the middle point; and
calculating a second postural value based on a difference between the third relative distance and the fourth relative distance.

15. The method of claim 1, wherein generating the graphical user interface comprises:
generating a digital representation of the user based on the first set of coordinates and the second set of coordinates; and
visually identifying a subset of anatomical landmarks on the digital representation that deviate from expected positions for the subset of anatomical landmarks.

16. The method of claim 1 further comprising:
presenting a notification from the musculoskeletal health evaluation system to a client device in response to determining that a subset of anatomical landmarks deviate from expected positions for the subset of anatomical landmarks.

17. The method of claim 16, wherein presenting the notification comprises one or more of providing haptic feedback or an audible sound on the client device.

18. A musculoskeletal health evaluation system comprising:
one or more hardware processors configured to:
receive an image of a user;
process the image by determining a first set of coordinates on the image associated with a first set of anatomical landmarks detected on a left side of the user and a second set of coordinates on the image associated with a second set of anatomical landmarks detected on a right side of the user;
measure asymmetry at corresponding regions of the left side and the right side of the user, wherein measuring the asymmetry comprises determining that the first set of coordinates for one or more anatomical landmarks from the first set of anatomical landmarks on the left side of the user deviate from the second set of coordinates for one or more corresponding anatomical landmarks from the second set of anatomical landmarks on the right side of the user; and
present a graphical user interface with an evaluation of musculoskeletal health based on the asymmetry that is measured at the corresponding regions of the user by the musculoskeletal health evaluation system.

19. A non-transitory computer-readable medium storing program instructions that, when executed by one or more hardware processors of a musculoskeletal health evaluation system, cause the musculoskeletal health evaluation system to perform operations comprising:
receiving an image of a user;
processing the image by determining a first set of coordinates on the image associated with a first set of anatomical landmarks detected on a left side of the user and a second set of coordinates on the image associated with a second set of anatomical landmarks detected on a right side of the user;
measuring asymmetry at corresponding regions of the left side and the right side of the user, wherein measuring the asymmetry comprises determining that the first set of coordinates for one or more anatomical landmarks from the first set of anatomical landmarks on the left side of the user deviate from the second set of coordinates for one or more corresponding anatomical landmarks from the second set of anatomical landmarks on the right side of the user; and
presenting a graphical user interface with an evaluation of musculoskeletal health based on the asymmetry that is measured at the corresponding regions of the user by the musculoskeletal health evaluation system.
